(19) United States
(12) Patent Application Publication
(10) Pub. No.: US 2007/0038036 A1
(43) Pub. Date: Feb. 15, 2007

(54) Method for determining a stress severity index

(76) Inventors: Orlando Sellers II, Peoria, IL (US); Pamela S. Saal, N. Pekin, IL (US)

Correspondence Address: CATERPILLAR INC.;100 N.E. ADAMS STREET, PATENT DEPT., PEORIA, IL, 616296490, US

(21) Appl. No.: 11/186,191

(22) Filed: Jul. 21, 2005

(51) Int. Cl. A61B 5/00

(52) U.S. Cl. 600/300

(57) ABSTRACT

A method for determining a stress severity index. The method includes determining a period of time engaged in an activity, determining a stress level index related to the activity, determining a cumulative exposure factor, and determining a stress severity index as a function of the period of time, the stress level index, and the cumulative exposure factor.

### TECHNICAL FIELD

This invention relates generally to a method for determining a stress severity index and, more particularly, to a method for comparing a determined stress severity index to a threshold to determine if further action is desired.

### BACKGROUND

Many activities are known to be associated with certain levels of physical stress. For example, certain types of jobs, sports activities, lifestyles, and the like, may be known to foster an environment that is conducive to stress. Furthermore, certain persons engaged in such activities may be more prone to the effects of stress than other individuals.

Although some instances of stress may be obvious to an observer, such as frequent repeated injury of a person, it is often difficult to recognize situations in which physical stress has exceeded desirable levels. The end result is failure to recognize stress related problems and taking actions to reduce stress before the problems become too severe.

The present invention is directed to overcoming one or more of the problems as set forth above.

### SUMMARY OF THE INVENTION

In one aspect of the present invention a method for determining a stress severity index is disclosed. The method includes determining a period of time engaged in an activity, determining a stress level index related to the activity, determining a cumulative exposure factor, and determining a stress severity index as a function of the period of time, the stress level index, and the cumulative exposure factor.

In another aspect of the present invention a method for determining a stress severity index of a worker is disclosed. The method includes determining a number of labor hours engaged by the worker over a period of time, determining a job stress level index, calculating a cumulative exposure factor, and calculating a stress severity index as a function of the number of labor hours, the job stress level index, and the cumulative exposure factor.

In another aspect of the present invention a system for determining a stress severity index is disclosed. The system includes a computer having an input and an output. The computer is configured to determine a period of time engaged in an activity, a stress level index related to the activity, a cumulative exposure factor, and a stress severity index as a function of the period of time, the stress level index, and the cumulative exposure factor.

In another aspect of the present invention a method for determining a stress severity index is disclosed. The method includes inputting information to a computer related to a period of time engaged in an activity, a stress level index, and a cumulative exposure factor. The method also includes receiving from the computer a normalized stress severity index determined from the inputted information, the normalized stress severity index being compared to a predetermined threshold. In addition, the method includes initiating an action to lower the normalized stress severity index in response to the comparison.

### BRIEF DESCRIPTION OF THE DRAWINGS

FIG. 1 is a block diagram illustrating a computer suited for use with the present invention;

FIG. 2 is a table depicting exemplary data associated with the present invention;

FIG. 3 is a graph illustrating life expectancy as a function of a stress severity index;

FIG. 4 is a flow diagram illustrating an aspect of the present invention; and

FIG. 5 is a flow diagram illustrating another aspect of the present invention.

### DETAILED DESCRIPTION

Referring to the drawings, a method for determining a stress severity index is shown. The present invention is described below with reference to determining a stress severity index of a worker at a job as an example. However, the present invention may be suited to a number of applications, including for example sports activities, medical applications, lifestyle analysis, and the like.

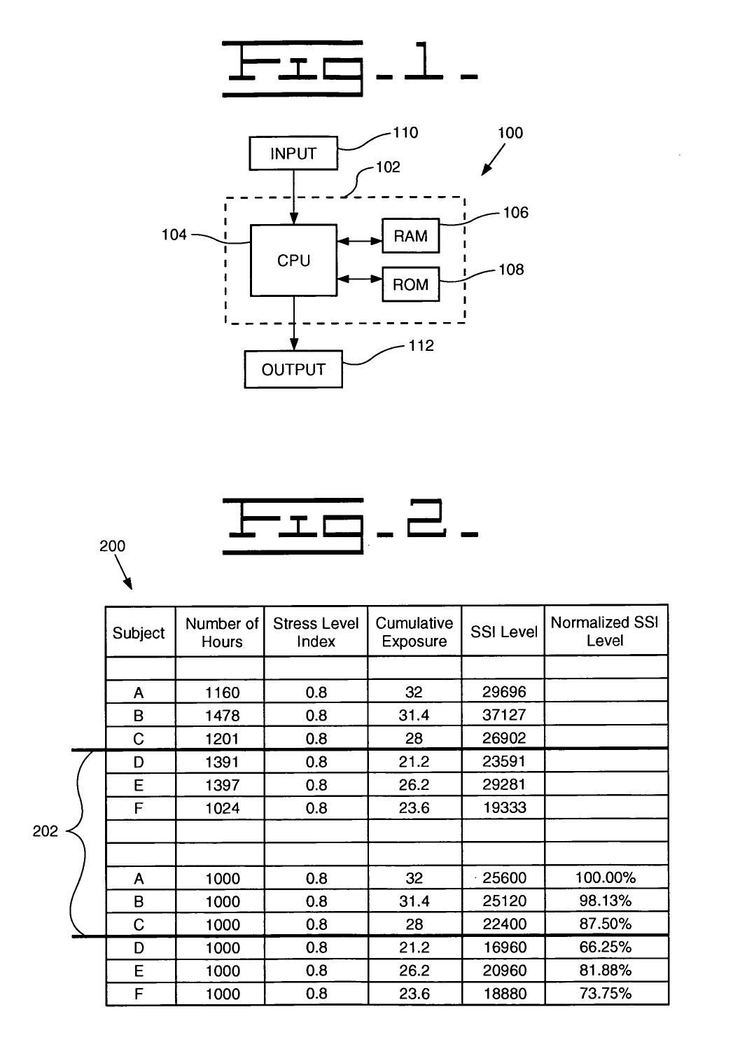

FIG. 1 illustrates a block diagram of a system 100 that may be used with the present invention. A computer 102 is linked to an input 110 and an output 112. The computer 102 may include a central processing unit (CPU) 104, and some form of memory storage, such as RAM 106, ROM 108, and the like. The computer 102 may include other components as well, such as is known in the art.

The input 110 may be any of a variety of devices used for inputting information to a computer. For example, the input 110 may include a keyboard, a mouse, a serial data link, and the like. Multiple combinations of inputs may be used.

The output 112 may be any of a variety of devices used for receiving information from a computer. For example, the output 112 may include a monitor, a printer, a serial data link, and the like. Multiple combinations of outputs may be used.

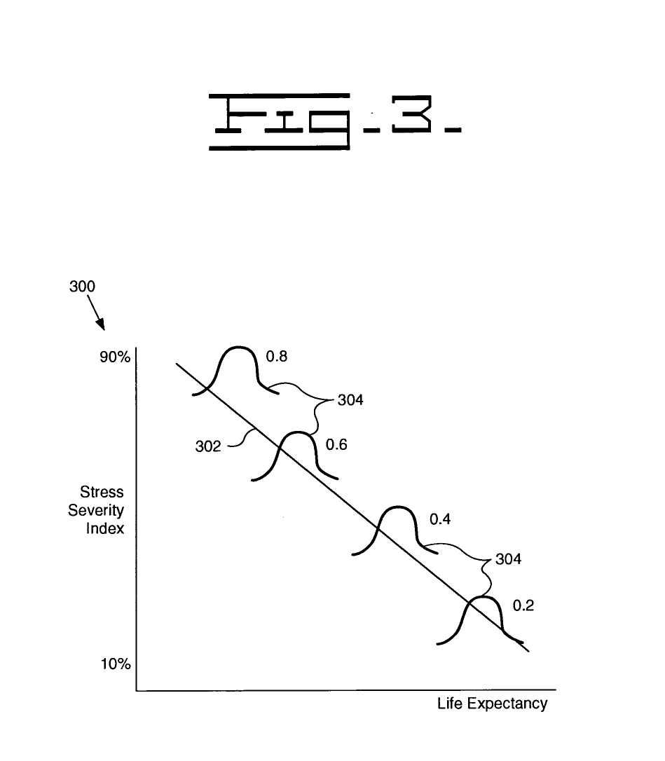

FIG. 2 illustrates a table 200 of exemplary stress severity index data. Six subjects, A-F, are being evaluated for their stress severity index levels. For exemplary purposes, the subjects may be workers who share a common stress level index; that is, they work at jobs exposing them to a similar amount of stress.

The number of hours column is indicative of a number of hours each worker performed the job, and may be based on a determined period of time. For example, the number of hours may be for a period of time of one recent calendar year. Worker A spent 1160 hours on the job during that year, worker B spent 1478 hours, worker C spent 1201 hours, and so on. In the bottom portion of the table 200, the number of hours is normalized to one number for all workers, for example 1000 hours, thus allowing for direct comparisons between all of the workers.

The stress level index is an industry standard determined by a standards group, such as the Presley Reed Medical Disability Advisor. The stress level index is known in the art, and is commonly used in other applications. For the present invention, the stress level index may be normalized to 1.0 and may have five categories, e.g., 0.2=sedentary, 0.4=light duty, 0.6=medium duty, 0.8=heavy duty, and 1.0=very heavy duty. As the stress level index increases, the likelihood of incurring a stress related incident, such as an injury, is greater. In the table 200, each of the workers A-F are exposed on the job to a stress level of index of 0.8, i.e., heavy duty.

Cumulative exposure is the weighted sum of the stress level of a worker's jobs over time multiplied by the time spent performing that job. For example, worker A may have had a prior job for 10 years that was classified as stress level index 0.8, another prior job for 20 years that was stress level index 0.6, and a current job for 15 years that is stress level index 0.8. The cumulative exposure for worker A would then be (10 * 0.8) +(20 * 0.6) +(15 * 0.8)=32.

The number of hours, current activity stress level index, and cumulative exposure for each worker are multiplied together to determine the stress severity index (SSI) level. Referring to the bottom portion of table 200, the SSI for worker A is 25600, the SSI for worker B is 25120, the SSI for worker C is 22400, and so on. Worker A has the highest SSI of all the evaluated workers. The normalized SSI level may be expressed as a percentage of a chosen reference. For example, worker A, the highest SSI, may be designated at 100%, and all other workers' SSI levels may be normalized as a percentage of that. Alternatively, other methods for normalizing may be used. For example, the worker having an SSI closest to an accepted threshold may be set as the 100% reference, and all other SSI levels may be based on that.

A predetermined threshold 202 may be established and is denoted in table 200 by lines through the cutoff between acceptable and unacceptable worker SSI levels. For example, workers A, B, and C may exceed the threshold 202, and workers D, E, and F may fall under the threshold 202. The predetermined threshold 202 may be determined from historic data. However, until sufficient historic data for determining an accurate threshold 202 is available, the threshold 202 may be roughly set based on one or more control subjects, e.g., workers D, E, and F, who are considered to have acceptable SSI levels. The threshold 202 may then be adjusted periodically as more historical data becomes available.

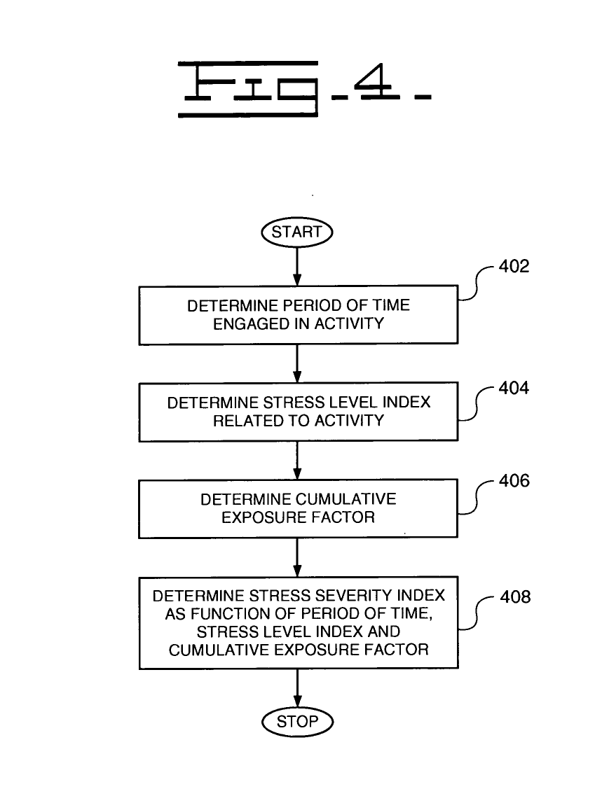

Referring to FIG. 3, a graph 300 of stress severity index vs. life expectancy is shown. The term life expectancy is used in the context of the time until a stress related incident occurs, and is not meant to denote an end of life experience.

A trend plot 302 is shown as a linear plot, but may be nonlinear as well, for example exponentially decreasing. The trend plot 302 represents the trend that a decreasing stress level index would result in a decreasing SSI. Along the trend plot 302, a series of stress level index plots 304 are shown. Each stress level index plot 304 represents the stress severity index vs. life expectancy for a particular environment having one stress level index, e.g., 0.8, 0.6, 0.4, 0.2. Each stress level index plot 304 is shown as a bell-type curve for exemplary purposes only. Each plot 304 may have its own unique curve characteristics. Various factors may determine the shape and location of each stress level index plot 304, for example job location, job type, and the like.

### INDUSTRIAL APPLICABILITY

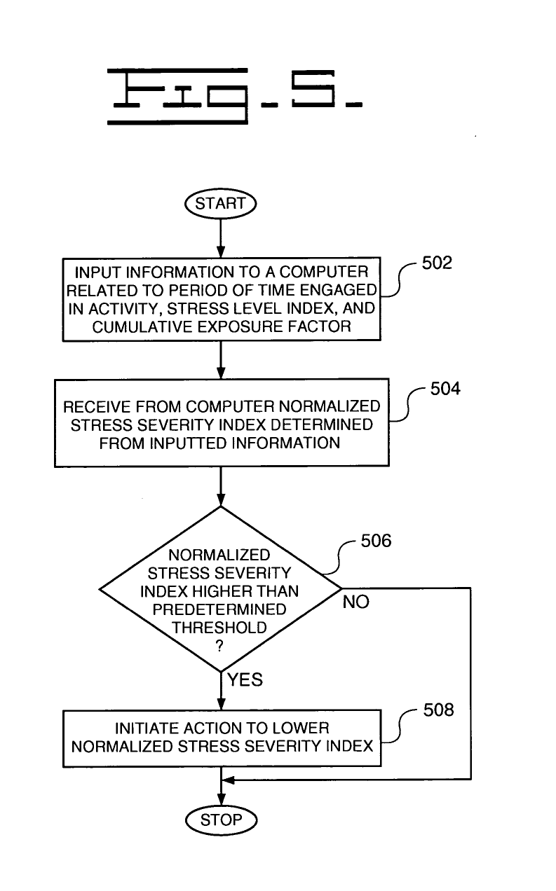

Application of the present invention may be described with reference to the flow diagrams of FIGS. 4 and 5.

In FIG. 4, in a first control block 402, a period of time an activity is engaged in is determined. Using the example situation of a worker at a job, the activity may be the job and the period of time may be a number of labor hours engaged by the worker at the job.

In a second control block 404, a stress level index related to the activity is determined. The stress level index may be a job stress level index determined from a standards organization, as is known in the art. Alternatively, an independent stress level index may be developed for use with the present invention. The stress level index exemplified above may be scaled as 0.2 for sedentary activities, 0.4 for light duty activities, 0.6 for medium duty activities, 0.8 for heavy duty activities, and 1.0 for very heavy duty activities.

In a third control block 406, a cumulative exposure factor is determined as a function of an accumulation of activities and associated stress levels. Determination of the cumulative exposure factor is described in detail above.

In a fourth control block 408, a stress severity index (SSI) is determined as a function of the period of time, the stress level index, and the cumulative exposure factor, i.e., by multiplying the three factors together. The SSI may be normalized to enable direct comparison among a group of subjects. The SSI may also be compared to a predetermined threshold 202. An SSI that is above the threshold 202 may indicate that action should be initiated to lower the SSI to an acceptable level.

Actions that may be initiated to lower an SSI that pertains to a physical stress level may include attention to ergonomics, cleanliness of the work environment, choice of proper tools, a stretching/exercise program initiated for the workers prior to the start of the work shift, and the like.

Although the present invention has been described above with respect to physical stress, in particular physical stress at a job site, application may be made to other types of stress, such as mental and emotional stress. An SSI for mental stress that is above an acceptable threshold may be lowered by initiating actions such as detailed training programs, providing counseling, addressing health and nutritional issues, and the like. An SSI for emotional stress that is above an acceptable threshold may be lowered by initiating actions such as re-organizing the work force to enhance compatibility among workers, initiating a team work environment, providing empowerment and showing trust in the workers, and the like.

The flow diagram of FIG. 5 further describes the present invention. In a first control block 502, information is input to a computer 102 related to a period of time engaged in an activity, a stress level index, and a cumulative exposure factor. The information may be entered manually or at least a portion of the information may be derived by the computer 102 from existing records in databases.

The computer 102 may then process the information and, in a second control block 504, a computer operator may receive a normalized stress severity index that is determined by the computer 102 in accordance with present invention as described above. The above steps may be used to determine an SSI for one individual or for each of a group of individuals, and the output from the computer 102 may be in any form desired, e.g., text, graphical, on a monitor, in a printout, and the like. In addition, the SSI may be incorporated into a database for historical and trend analysis.

In a decision block 506, the normalized SSI is compared to a predetermined threshold 202. If the SSI is lower than the threshold 202, then a decision that no action is needed may be made. However, if the SSI is higher than the threshold 202, then control proceeds to a third control block 508 and a decision to initiate some type of action to lower the SSI may be made.

Other aspects can be obtained from a study of the drawings, the disclosure, and the appended claims.

What is claimed is:

1. A method for determining a stress severity index, comprising the steps of:
determining a period of time engaged in an activity;
determining a stress level index related to the activity;
determining a cumulative exposure factor; and
determining a stress severity index as a function of the period of time, the stress level index, and the cumulative exposure factor.

2. A method, as set forth in claim 1, further including the step of determining a normalized stress severity index as a function of the stress severity index.

3. A method, as set forth in claim 2, further including the step of comparing the normalized stress severity index to a predetermined threshold indicative of an acceptable stress severity index.

4. A method, as set forth in claim 3, further including the step of initiating an action in response to the normalized stress severity index exceeding the predetermined threshold.

5. A method, as set forth in claim 1, wherein determining a period of time engaged in an activity includes the step of determining a period of time of a person engaged in the activity.

6. A method, as set forth in claim 5, wherein determining a period of time includes the step of determining a total amount of activity engagement time during a time span.

7. A method, as set forth in claim 1, wherein determining a stress level index includes the step of selecting a stress level index from a predetermined standard.

8. A method, as set forth in claim 1, wherein determining a cumulative exposure factor includes the step of calculating a cumulative exposure factor as a function of an accumulation of activities and associated stress levels.

9. A method, as set forth in claim 4, wherein initiating an action includes the step of initiating an action to lower the stress severity index.

10. A method for determining a stress severity index of a worker, comprising the steps of:
determining a number of labor hours engaged by the worker over a period of time;
determining a job stress level index;
calculating a cumulative exposure factor; and
calculating a stress severity index as a function of the number of labor hours, the job stress level index, and the cumulative exposure factor.

11. A method, as set forth in claim 10, further including the steps of:
normalizing the stress severity index;
comparing the normalized stress severity index to a predetermined threshold that is indicative of an acceptable stress severity index; and
initiating at least one action to lower the normalized stress severity index in response to the normalized stress severity index being higher than the predetermined threshold.

12. A system for determining a stress severity index; comprising:
a computer having an input and an output;
wherein the computer is configured to determine a period of time engaged in an activity, a stress level index related to the activity, a cumulative exposure factor, and a stress severity index as a function of the period of time, the stress level index, and the cumulative exposure factor.

13. A system, as set forth in claim 12, wherein the computer is further configured to determine a normalized stress severity index as a function of the stress severity index, compare the normalized stress severity index to a predetermined threshold indicative of an acceptable stress severity index, and provide the results of the comparison.

14. A method for determining a stress severity index, comprising the steps of:
inputting information to a computer related to a period of time engaged in an activity, a stress level index, and a cumulative exposure factor;
receiving from the computer a normalized stress severity index determined from the inputted information, the normalized stress severity index being compared to a predetermined threshold; and
initiating an action to lower the normalized stress severity index in response to the comparison.
